(19) United States
(12) Patent Application Publication
(10) Pub. No.: US 2022/0378581 A1
(43) Pub. Date: Dec. 1, 2022

(54) TIBIAL PROSTHESIS AND KNEE PROSTHESIS

(71) Applicant: SUZHOU MICROPORT ORTHORECON CO., LTD., Suzhou (CN)

(72) Inventors: Huanhuan HUANG, Suzhou (CN); Yandong SUN, Suzhou (CN); Kaiyu ZHAO, Suzhou (CN); Chih-Hsin WENG, Suzhou (CN)

(21) Appl. No.: 17/773,743

(22) PCT Filed: Sep. 25, 2020
(86) PCT No.: PCT/CN2020/117950

§ 371 Date: May 2, 2022

(30) Foreign Application Priority Data
Oct. 31, 2019 (CN) 201911053045.4

(51) Int. Cl. A61F 2/38; A61F 2/30

(52) U.S. Cl. CPC A61F 2/389; A61F 2/30734; A61F 2002/30131; A61F 2002/30113

(57) ABSTRACT

A tibial prosthesis and a knee prosthesis are provided. The tibial prosthesis includes a tibial plateau, a first tibial insert and a second tibial insert. The tibial plateau has a proximal end face defining a mount surface arranged with respect to a first axis. The first tibial insert is disposed on the mount surface rotatably about the first axis. The first tibial insert has a first concave surface centered on the first axis. The first concave surface is distally recessed. The second tibial insert is disposed on the tibial plateau and has a second concave surface extending along an arc-shaped trajectory line that is parallel to the mount surface. The arc-shaped trajectory line is centered on the first axis, and the second concave surface is distally recessed. The first tibial insert and the second tibial insert are arranged independently of each other, and the first tibial insert is located medially to the second tibial insert.

### TECHNICAL FIELD

The present invention relates to the field of medical devices, and in particular, to a tibial prosthesis and a knee prosthesis.

### BACKGROUND

In existing mobile bearing knee prostheses, a tibial insert is typically secured to the tibial plateau by inserting a downward extending stem of the tibial insert into a central hole made in the tibial plateau so that the tibial insert is radially restricted while being rotatable about a central axis. However, the rotation achieved by this design differs considerably from the medial pivot motion of the normal knee. The medial pivot motion refers to rolling of the femoral condyles on the tibial plateau during transitions between extension and flexion of the knee, which consists of rotation of the lateral femoral condyle on the lateral articular surface of the tibial plateau about the center of a ball and socket joint defined by the medial femoral condyle and the medial articular surface of the tibial plateau. In order to overcome this, some of the traditional mobile bearing prostheses were modified to medially shift the axis of rotation of the tibial insert to allow medial rotation movement of the tibial insert and the tibial plateau within an expanded range of medial and lateral rotation. These modified versions are termed as medial pivot mobile bearing knee prostheses. Since the human knee favors mechanically acting on the medial side of an articular surface, shifting the axis of rotation medially facilitates the bearing of pressure from the human body itself as well as shear forces exerted on the joint during movements, so it seems somewhat beneficial for the stability of those prostheses. However, it also leads to more frequent and more vigorous up-and-down swinging movement of a lateral portion of the tibial insert, tending to cause dislocation of the insert from the plateau if restriction from the axis of rotation is insufficient. From this point of view, it would be adverse to the stability of the prostheses.

There are also medial pivot fixed bearing knee prostheses in which medial rotation movement is enabled usually by restriction by a medial articular surface of a tibial insert and relative rotation of a lateral articular surface. However, this approach often involves large relative displacement of the tibial insert from the femoral condyles, which increases the risk of surface wear of the knee prosthesis and the femoral condyles and leads to a higher revision rate.

### SUMMARY OF THE INVENTION

It is an objective of the present invention to overcome the problem of easy surface wear with existing knee prostheses by presenting a tibial prosthesis and a novel knee prosthesis.

To this end, the tibial prosthesis provided in the present invention includes:

a tibial plateau having a proximal end face defining a mount surface arranged with respect to a first axis;

a first tibial insert disposed on the mount surface rotatably about the first axis in such a manner that a normal of the mount surface and the first axis form an angle in the range of 0°-5°, the first tibial insert having a first concave surface which is centered on the first axis and distally recessed; and

a second tibial insert disposed on the tibial plateau, the second tibial insert having a second concave surface extending along an arc-shaped trajectory line that is parallel to the mount surface, the arc-shaped trajectory line centered on the first axis, the second concave surface recessed distally,

wherein the first tibial insert and the second tibial insert are arranged independently of each other, and the first tibial insert is located medially to the second tibial insert.

Optionally, the second tibial insert may be detachably attached to the tibial plateau.

Optionally, one of a distal end face of the second tibial insert and the proximal end face of the tibial plateau may be provided with a channel, with the other being provided with a collar engageable with the channel, wherein the second tibial insert is attached to the tibial plateau by engaging the channel with the collar along a direction perpendicular to the first axis.

Optionally, the channel may be tapered or rectangular.

Optionally, one of the mount surface of the tibial plateau and the distal end face of the first tibial insert may be provided with a mount hole, with the other being provided with a mount stud engageable with the mount hole, wherein both the mount stud and the mount hole define axes that coincide with the first axis, and the mount stud is inserted in the mount hole rotatably about the first axis.

Optionally, both the mount stud and the mount hole may be cylindrical.

Optionally, the tibial plateau may include a stem defining an axis parallel to the first axis, the stem disposed at a distal end of the tibial plateau.

Optionally, the first concave surface may be a spherical surface with its spherical center located on the first axis.

Optionally, the first tibial insert may have a circular cross section.

To the above end, the knee prosthesis provided in the present invention includes a femoral condyle prosthesis and the tibial prosthesis as defined above, the femoral condyle prosthesis including a medial condyle and a lateral condyle, the first tibial insert of the tibial prosthesis corresponding to the medial condyle, the second tibial insert of the tibial prosthesis corresponding to the lateral condyle, wherein relative flexion movement between the femoral condyle prosthesis and the tibial prosthesis consists of rotation of the medial condyle relative to the first tibial insert concurrently with rotation of the first tibial insert relative to the tibial plateau and rolling of the lateral condyle on the second tibial insert concurrently with the second tibial insert being kept stationary relative to the tibial plateau.

In summary, the present invention provides a tibial prosthesis and a knee prosthesis. The tibial prosthesis includes a tibial plateau, a first tibial insert and a second tibial insert. The tibial plateau has a proximal end face defining a mount surface. The first tibial insert is disposed on the mount surface rotatably about a first axis in such a manner that a normal of the mount surface and the first axis form an angle in the range of 0°-5°. The first tibial insert has a first concave surface, which is centered on the first axis and distally recessed. The second tibial insert is disposed on the tibial plateau and has a second concave surface extending along an arc-shaped trajectory line that is parallel to the mount surface. The arc-shaped trajectory line is centered on the first axis, and the second concave surface is recessed distally. The first tibial insert and the second tibial insert are arranged independently of each other, and the first tibial insert is located medially to the second tibial insert. The two independent tibial inserts correspond respectively to the medial and lateral condyles. Specifically, the first tibial insert corresponds to the medial condyle and the second tibial insert to the lateral condyle. The first tibial insert is rotatable relative to the tibial plateau, while the second tibial insert is kept stationary relative to the tibial plateau, ensuring relative stability of the lateral condyle. In this way, during medial rotation movement of the femoral condyle and the tibial prosthesis, double rotational motion of the medial condyle (consisting of rotation of the medial condyle relative to the first tibial insert and rotation of the first tibial insert relative to the tibial plateau) is made possible, which reduces the risk of wear of the tibial prosthesis.

### BRIEF DESCRIPTION OF THE DRAWINGS

Those of ordinary skill in the art would appreciate that the following drawings are presented merely to enable a better understanding of the present invention rather than to limit the scope thereof in any sense. In the drawings:

FIG. 1 is a schematic overview of a tibial prosthesis according to an embodiment of the present invention;

FIG. 2 is a schematic illustration of a tibial plateau according to an embodiment of the present invention;

FIG. 3 is a schematic illustration of a first tibial insert according to an embodiment of the present invention;

FIG. 4 is an exploded view of the tibial prosthesis according to an embodiment of the present invention;

FIG. 5 is a top view of the tibial prosthesis according to an embodiment of the present invention;

FIG. 6 is a schematic illustration of a second tibial insert according to an embodiment of the present invention;

FIG. 7 is a schematic axial cross-sectional view of the tibial prosthesis according to an embodiment of the present invention;

FIGS. 8a and 8b schematically illustrate possible forms of a channel according to embodiments of the present invention; and

FIGS. 9a to 9d schematically illustrate possible forms of a mount hole and a mount stud according to embodiments of the present invention.

In these figures,
1-tibial plateau; 1a-mount surface; 1b-stem; 1a1-channel; 1a2-mount hole;
2-first tibial insert; 2a-first concave surface; 2b-mount stud; 2bc-first axis;
3-second tibial insert; 3a-second concave surface; 3ac-center of an arc-shaped trajectory line; 3at-arc-shaped trajectory line; 3b-collar.

### DETAILED DESCRIPTION

Objects, features and advantages of the present invention will become more apparent upon reading the following more detailed description of the present invention, which is set forth by way of particular embodiments with reference to the accompanying drawings. Note that the figures are provided in a very simplified form not necessarily drawn to exact scale and for the only purpose of facilitating easy and clear description of the embodiments. In addition, structures shown in the figures are usually partially representations of their actual counterparts. In particular, as the figures would have different emphases, they are sometimes drawn to different scales.

As used herein and in the appended claims, the singular forms “a”, “an” and “the” include plural referents, unless the context clearly dictates otherwise. As used herein and in the appended claims, the term “or” is generally employed in the sense of “and/or”, unless the context clearly dictates otherwise. In general, the term “medial” generally refers to a side closer to the median sagittal plane of the human body, while the term “lateral” generally refers to a side farther away from the median sagittal plane of the human body. As used herein, the terms “left” and “right” refer to left and right sides of a patient's body mentioned with respect to an orientation in which a prosthesis is implanted into the body. The term “proximal” refers to an end closer to the human heart, whilst the term “distal” refers to an end farther from the human heart.

The core idea of the present invention is to provide a tibial prosthesis and a knee prosthesis, the tibial prosthesis including a tibial plateau, a first tibial insert and a second tibial insert, the tibial plateau having a proximal end face defining a mount surface, the first tibial insert being disposed on the mount surface rotatably about a first axis in such a manner that a normal of the mount surface and the first axis form an angle in the range of 0°-5°, the first tibial insert having a first concave surface, which is centered on the first axis and distally recessed, the second tibial insert being disposed on the tibial plateau and having a second concave surface extending along an arc-shaped trajectory line that is parallel to the mount surface, the arc-shaped trajectory line being centered on the first axis, the second concave surface being recessed distally. In addition, the first tibial insert and the second tibial insert are arranged independently of each other, and the first tibial insert is located medially to the second tibial insert. The two independent tibial inserts correspond respectively to the medial and lateral condyles. Specifically, the first tibial insert corresponds to the medial condyle and the second tibial insert to the lateral condyle. The first tibial insert is rotatable relative to the tibial plateau, while the second tibial insert is kept stationary relative to the tibial plateau, ensuring relative stability of the lateral condyle. In this way, during medial rotation movement of the femoral condyle and the tibial prosthesis, double rotational motion of the medial condyle (consisting of rotation of the medial condyle relative to the first tibial insert and rotation of the first tibial insert relative to the tibial plateau) is made possible, which reduces the risk of wear of the tibial prosthesis.

Further description is set forth below with reference to the accompanying drawings.

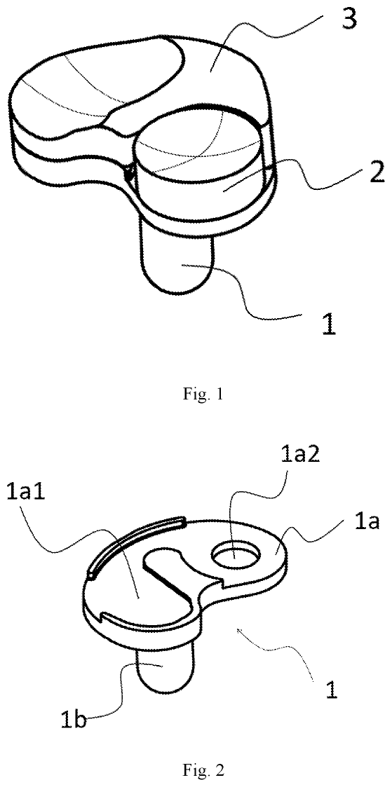

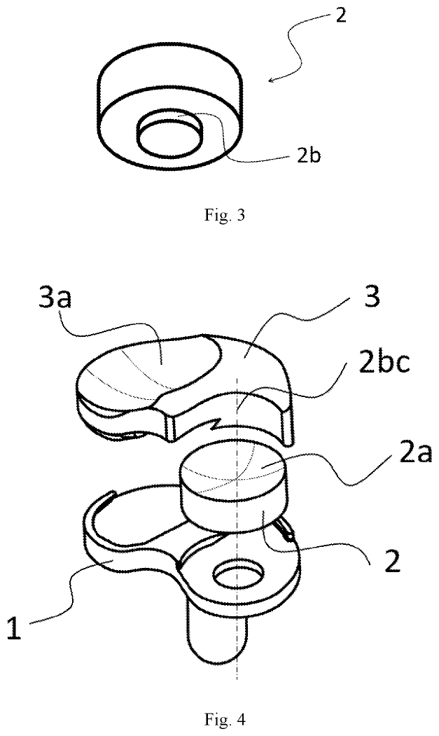

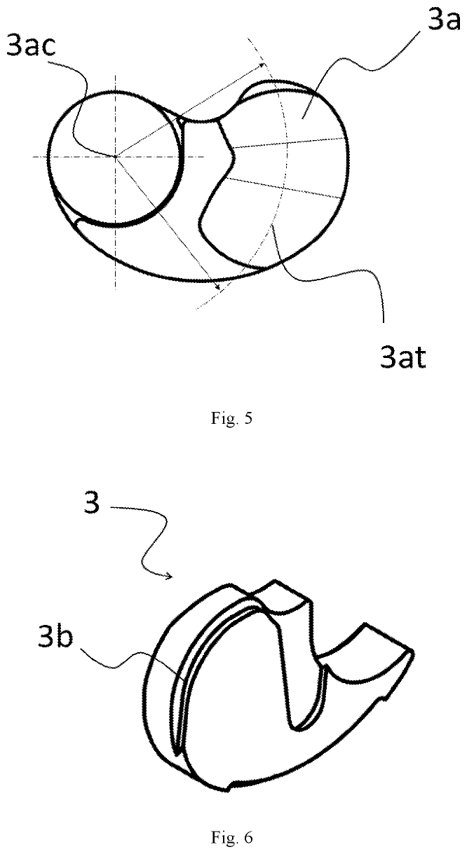

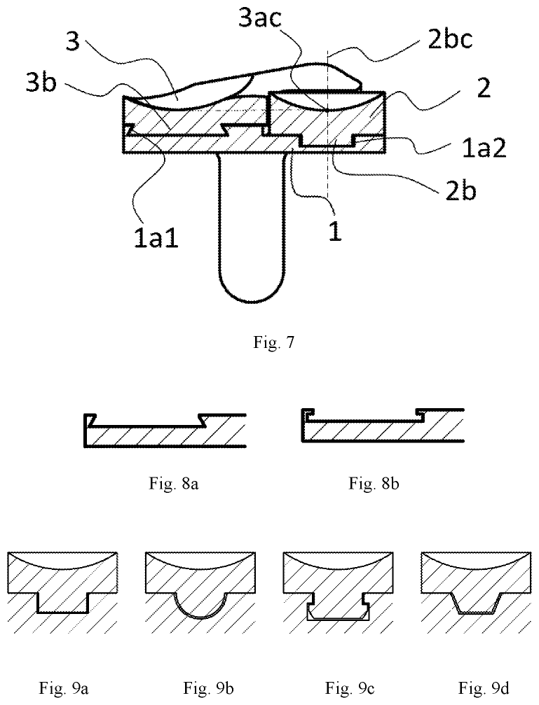

Reference is now made to FIGS. 1 to 7, 8a and 8b and 9a to 9d. FIG. 1 is a schematic overview of a tibial prosthesis according to an embodiment of the present invention. FIG. 2 is a schematic illustration of a tibial plateau according to an embodiment of the present invention. FIG. 3 is a schematic illustration of a first tibial insert according to an embodiment of the present invention. FIG. 4 is an exploded view of the tibial prosthesis according to an embodiment of the present invention. FIG. 5 is a top view of the tibial prosthesis according to an embodiment of the present invention. FIG. 6 is a schematic illustration of a second tibial insert according to an embodiment of the present invention. FIG. 7 is a schematic axial cross-sectional view of the tibial prosthesis according to an embodiment of the present invention. FIGS. 8a and 8b schematically illustrate possible forms of a channel according to embodiments of the present invention. FIGS. 9a to 9d schematically illustrate possible forms of a mount hole and a mount stud according to embodiments of the present invention.

As shown in FIGS. 1, 4 and 5, according to embodiments of the present invention, the tibial prosthesis includes the tibial plateau 1, the first tibial insert 2 and the second tibial insert 3. A proximal end face of the tibial plateau 1 defines a mount surface 1a arranged with respect to a first axis. That is, the mount surface 1a faces the human femur (shown as facing upward in the figures). The first tibial insert 2 is disposed on the mount surface 1a rotatably about the first axis 2bc. The first axis 2bc passes a location of the tibial plateau 1 for interaction with the medial condyle and serves as an axis of rotation for medial rotation movement of the prosthesis. It is to be noted that the first axis 2bc is maintained substantially perpendicular to the mount surface 1a. That is, a normal of the mount surface 1a forms an angle in the range of 0° to 5° with the first axis 2bc (i.e., the angle between the normal of the mount surface 1a and the first axis 2bc is greater than or equal to 0° and smaller than or equal to 5°). Preferably, the first axis 2bc is perpendicular to the mount surface 1a. The first tibial insert 2 has a first concave surface 2a centered on the first axis 2bc. The first concave surface 2a is distally recessed and preferred to be a spherical surface (ball socket like articular surface) with its spherical center being located on the first axis 2bc. The second tibial insert 3 is disposed on the tibial plateau 1 and has a second concave surface 3a second, the concave surface 3a extends along an arc-shaped trajectory line 3at which is parallel to the mount surface 1a and centered 3ac on the first axis 2bc. The second concave surface 3a is recessed distally.

The first tibial insert 2 and the second tibial insert 3 are independently arranged side by side in a transverse direction. The first tibial insert 2 is located medially to the second tibial insert 3. The two independent tibial inserts (i.e., the first tibial insert 2 and the second tibial insert 3) correspond respectively to the medial and lateral condyles. Specifically, the first tibial insert 2 corresponds to the medial condyle, and the second tibial insert 3 to the lateral condyle. The first tibial insert 2 is rotatable relative to the tibial plateau 1, imparting a greater degree of freedom to the knee joint and greater fitness with the tibia during medial rotation movement to the tibial prosthesis. The second tibial insert 3 is kept stationary relative to the tibial plateau, ensuring relative stability of the lateral condyle and safety of the tibial prosthesis. In practical use, the first concave surface 2a of the first tibial insert 2 interacts with the medial femoral condyle, allowing rotational motion of the medial condyle consisting of rotation of the medial condyle about the first axis 2bc relative to the first tibial insert 2 and rotation of the first tibial insert 2 about the first axis 2bc relative to the tibial plateau 1. In addition, the second concave surface 3a of the second tibial insert 3 interacts with the lateral femoral condyle, enabling rolling of the lateral condyle along the arc-shaped trajectory line 3at of the second tibial insert 3 while an angle of flexion of the knee is varying. Since the center 3ac of the arc-shaped trajectory line 3at is also located on the first axis 2bc, centers of rotation for the medial and lateral condyles coincide with each other within a medial weight-bearing region of the knee joint. This better mimics the natural medial pivot motion of the normal human knee, imparting improved stability to the tibial prosthesis. The double rotational motion of the medial condyle (consisting of rotation of the medial condyle relative to the first tibial insert 2 and rotation of the first tibial insert 2 relative to the tibial plateau 1) during medial rotation movement of the femoral condyle and the tibial prosthesis splits friction of the medial condyle and the tibial insert between the rotational degree of freedom of the medial condyle relative to the first tibial insert 2 and the rotational degree of freedom of the first tibial insert 2 relative to the tibial plateau 1, thus lowering the risk of wear of the tibial prosthesis.

It will be appreciated that while the tibial prosthesis is illustrated in FIG. 1 as having a configuration suitable for use in a human right knee, it may be adapted for use in a human left knee by adopting a configuration mirrored to that of FIG. 1. In addition, the tibial prosthesis may be made in various sizes to meet the needs of different patients. Optional, referring to FIG. 2, the tibial plateau 1 may include a stem 1b defining an axis parallel to the first axis 2bc, which is disposed at a distal end of the tibial plateau. The stem 1b may distally extend from the mount surface 1a. In practice, the stem 1b is intended to be implanted and fixed within a patient's tibia. Preferably, the stem is cylindrical and defines a smooth ball at its distal end, which facilitates surgical implantation in the tibia.

Preferably, the second tibial insert 3 is detachably attached to the tibial plateau 1. This allows a physician to select suitable tibial insert/tibial plateau combinations for various anatomical structures and sizes of different patients. Optionally, one of a distal end face of the second tibial insert 3 and the proximal end face of the tibial plateau 1 may be provided with a channel 1a1, with the other being provided with a collar 3b engageable with the channel 1a1. Engaging the channel 1a1 with the collar 3b in a direction perpendicular to the first axis may result in attachment of the second tibial insert 3 to the tibial plateau 1. As shown in FIGS. 2, 7, 8a and 8b, in an exemplary embodiment, the channel 1a1 is provided in the proximal end face of the tibial plateau 1 (substantially along a peripheral edge of the mount surface 1a in the vicinity of a lateral region of the tibial plateau 1), while the collar 3b is provided on the distal end face of the second tibial insert 3. By engaging the channel 1a1 with the collar 3b, the second tibial insert 3 can be reliably and fixedly attached (i.e., locked) to the tibial plateau 1, without either axially or circumferential movement therebetween, thus ensuring desired stability of the second tibial insert 3. Preferably, the engagement of the channel 1a1 with the collar 3b is accomplished along a direction perpendicular to the first axis 2bc. That is, the second tibial insert 3 is inserted radially with respect to the tibial plateau 1 and thus attached thereto. Referring to FIGS. 8a and 8b, optionally, the channel may assume any of several forms. FIG. 8a is a schematic cross-sectional view of the channel in a tapered form. FIG. 8b is a schematic cross-sectional view of the channel in a rectangular form. Of course, other forms of the channel are also possible, such as semicircular. The collar 3b is complementary in shape to the channel 1a1, in order for close engagement to be achieved therebetween. It will be appreciated that, in some other embodiments, the channel 1a1 may be provided at the bottom of the second tibial insert 3, while the collar 3b may be provided on the tibial plateau 1. In such embodiments, attachment of the second tibial insert 3 to the tibial plateau 1 can also be accomplished as a result of engagement of the two components. In still some other embodiments, the second tibial insert 3 may be detachably attached to the tibial plateau 1 otherwise, for example, by snapping or bolting. The present invention is not limited to any particular method of establishing the datable attachment.

Additionally, one of the mount surface 1a of the tibial plateau 1 and the distal end face of the first tibial insert 2 may be provided with a mount hole 1a2, with the other being provided with a mount stud 2b engageable with the mount hole 1a2. Both axes of the mount stud 2b and the mount hole 1a2 may coincide with the first axis 2bc, and the mount stud 2b may inserted in the mount hole 1a2 rotatably about the first axis 2bc. Referring to FIGS. 2 to 4, in connection with FIG. 7, in an exemplary embodiment, the mount hole 1a2 is provided in the mount surface 1a of the tibial plateau 1, while the mount stud 2b is provided at the bottom of the first tibial insert 2 (i.e., on the distal end face). An outer size of the mount stud 2b may be slightly smaller than an inner size of the mount hole 1a2, in order to facilitate insertion and rotation of the mount stud 2b in the mount hole 1a2. Coincidence of the axis of the mount stud 2b with the first axis 2bc enables concentric rotation of the first concave surface 2a with eccentricity with respect to the first axis 2bc when the first tibial insert 2 is rotating about the first axis 2bc relative to the tibial plateau 1. FIGS. 9a to 9d schematically illustrate multiple possible forms of the mount stud 2b and the mount hole. In the case of FIG. 9a, both the mount stud 2b and the mount hole 1a2 are cylindrical. In the case of FIG. 9b, both the mount stud 2b and the mount hole 1a2 are hemispherical. In the case of FIG. 9c, the mount stud 2b and the mount hole 1a2 are partially raised and recessed, respectively. In the case of FIG. 9d, both the mount stud 2b and the mount hole 1a2 are tapered. All of them forms enable engagement of the mount stud 2b and the mount hole 1a2 in such a manner that the mount stud 2b is limited by the mount hole 1a2 from moving radially and axially while being allowed to circumferentially move relative to the mount hole 1a2. It is to be noted that the forms of the mount stud 2b and the mount hole 1a2 shown in FIGS. 9a to 9d are illustrative rather than limiting, and other forms are also possible, such as dovetail-shaped (E-shaped) or conical. It will be appreciated that, in some other embodiments, the mount hole 1a2 is provided in the first tibial insert 2 and the mount stud 2b on the mount surface 1a of the tibial plateau 1. Such embodiments also allow rotatable attachment of the first tibial insert 2 to the tibial plateau 1. Preferably, both the mount stud 2b and the mount hole 1a2 are cylindrical for the sake of easy engagement and structural simplicity. Optionally, the first tibial insert 2 may have a circular cross section, which avoids the first tibial insert 2 from coming into contact with the second tibial insert 3 during its rotation relative to the tibial plateau 1.

The present invention further provides a knee prosthesis including a femoral condyle prosthesis and the tibial prosthesis as defined above. The femoral condyle prosthesis includes a medial condyle and a lateral condyle. The first tibial insert corresponds to the medial condyle and the second tibial insert to the lateral condyle. Relative flexion movement between the femoral condyle prosthesis and the tibial prosthesis consists of rotation of the medial condyle relative to the first tibial insert concurrently with rotation of the first tibial insert relative to the tibial plateau and rolling of the lateral condyle on the second tibial insert concurrently with the second tibial insert being kept stationary relative to the tibial plateau. Since the knee prosthesis incorporates the tibial prosthesis as defined above, it also has the same benefits of the tibial prosthesis. Those skilled in the art can properly configure the femoral condyle prosthesis in the knee prosthesis as is known in the art, and a detailed description thereof is deemed unnecessary and is therefore omitted.

In summary, the present invention provides a tibial prosthesis and a knee prosthesis. The tibial prosthesis includes a tibial plateau, a first tibial insert and a second tibial insert. The tibial plateau has a proximal end face defining a mount surface. The first tibial insert is disposed on the mount surface rotatably about a first axis in such a manner that a normal of the mount surface and the first axis form an angle in the range of 0°-5°. The first tibial insert has a first concave surface, which is centered on the first axis and distally recessed. The second tibial insert is disposed on the tibial plateau and has a second concave surface extending along an arc-shaped trajectory line that is parallel to the mount surface. The arc-shaped trajectory line is centered on the first axis, and the second concave surface is recessed distally. The first tibial insert and the second tibial insert are arranged independently of each other, and the first tibial insert is located medially to the second tibial insert. The two independent tibial inserts correspond respectively to the medial and lateral condyles. Specifically, the first tibial insert corresponds to the medial condyle and the second tibial insert to the lateral condyle. The first tibial insert is rotatable relative to the tibial plateau, while the second tibial insert is kept stationary relative to the tibial plateau, ensuring relative stability of the lateral condyle. In this way, during medial rotation movement of the femoral condyle and the tibial prosthesis, double rotational motion of the medial condyle (consisting of rotation of the medial condyle relative to the first tibial insert and rotation of the first tibial insert relative to the tibial plateau) is made possible, which reduces the risk of wear of the tibial prosthesis.

The foregoing description presents merely some preferred embodiments of the present invention and is not intended to limit the scope of the present invention in any sense. It is intended that all changes and modifications made by those of ordinary skill in the art in light of the above teachings fall within the scope of the appended claims.

1. A tibial prosthesis, comprising:
a tibial plateau having a proximal end face defining a mount surface;
a first tibial insert disposed on the mount surface rotatably about a first axis in such a manner that a normal of the mount surface and the first axis form an angle in the range of 0°-5°, the first tibial insert having a first concave surface which is centered on the first axis and recessed towards a distal end thereof; and
a second tibial insert disposed on the tibial plateau, the second tibial insert having a second concave surface extending along an arc-shaped trajectory line that is parallel to the mount surface, the arc-shaped trajectory line centered on the first axis, the second concave surface recessed towards a distal end thereof,
wherein the first tibial insert and the second tibial insert are arranged independently of each other, and the first tibial insert is located medially to the second tibial insert.

2. The tibial prosthesis according to claim 1, wherein the second tibial insert is detachably connected to the tibial plateau.

3. The tibial prosthesis according to claim 2, wherein one of a distal end face of the second tibial insert and the proximal end face of the tibial plateau is provided with a channel, with the other being provided with a collar engageable with the channel, and wherein the second tibial insert is connected to the tibial plateau by engaging the channel with the collar along a direction perpendicular to the first axis.

4. The tibial prosthesis according to claim 3, wherein the channel is tapered or rectangular.

5. The tibial prosthesis according to claim 1, wherein one of the mount surface of the tibial plateau and the distal end face of the first tibial insert is provided with a mount hole, with the other being provided with a mount stud engageable with the mount hole, both the mount stud and the mount hole defining axes that coincide with the first axis, the mount stud inserted in the mount hole rotatably about the first axis.

6. The tibial prosthesis according to claim 5, wherein both the mount stud and the mount hole are cylindrical.

7. The tibial prosthesis according to claim 1, wherein the tibial plateau comprises a stem defining an axis parallel to the first axis, the stem disposed at a distal end of the tibial plateau.

8. The tibial prosthesis according to claim 1, wherein the first concave surface is a spherical surface, the spherical surface having a spherical center located on the first axis.

9. The tibial prosthesis according to claim 1, wherein the first tibial insert has a circular cross section.

10. A knee prosthesis, comprising a femoral condyle prosthesis and the tibial prosthesis according to claim 1, the femoral condyle prosthesis comprising a medial condyle and a lateral condyle, the first tibial insert of the tibial prosthesis corresponding to the medial condyle, the second tibial insert of the tibial prosthesis corresponding to the lateral condyle, wherein relative flexion movement between the femoral condyle prosthesis and the tibial prosthesis consists of rotation of the medial condyle relative to the first tibial insert concurrently with rotation of the first tibial insert relative to the tibial plateau and rolling of the lateral condyle on the second tibial insert concurrently with the second tibial insert being kept stationary relative to the tibial plateau.
